(19) United States
(12) Patent Application Publication
(10) Pub. No.: US 2006/0188523 A1
(43) Pub. Date: Aug. 24, 2006

(54) Methods for diagnosing and treating chronic tonsillitis

(76) Inventors: Zhiheng Pei, New York, NY (US); Gurdip Sidhu, Harrington Park, NJ (US); Liying Yang, New York, NY (US); Pranil Chandra, New York, NY (US); Xiaohua Lu, Croton on Hudson, NY (US); Anna Pei, New York, NY (US)

Correspondence Address: KLAUBER & JACKSON, 411 HACKENSACK AVENUE, HACKENSACK, NJ, 07601, US

(21) Appl. No.: 11/328,727

(22) Filed: Jan. 10, 2006

Related U.S. Application Data
(60) Provisional application No. 60/642,609, filed on Jan. 10, 2005.

(51) Int. Cl. A61K 39/02; G01N 33/554

(52) U.S. Cl. 424/234.1; 435/7.32

(57) ABSTRACT

The invention relates to diagnostic methods, therapeutic methods and kits for diagnosing and treating chronic adenotonsillar tonsillitis and adenotonsillar hypertrophy as well as vaccines for preventing chronic adenotonsillar tonsillitis and adenotonsillar hypertrophy.

The present application claims priority to U.S. Provisional Patent Application Ser. No. 60/642,609 filed on Jan. 10, 2005.

### FIELD OF THE INVENTION

The invention relates to diagnostic methods, therapeutic methods and kits for diagnosing and treating chronic adenotonsillar tonsillitis and adenotonsillar hypertrophy as well as vaccines for preventing chronic adenotonsillar tonsillitis and adenotonsillar hypertrophy.

### BACKGROUND OF THE INVENTION

Chronic adenotonsillar hypertrophy/tonsillitis (CATH/T) refers to a chronic enlargement of the tonsils and/or adenoids with or without episodic acute or chronic inflammation. Chronic adenotonsillar hypertrophy and tonsillitis is an obstructive lymphoproliferative disease that affects children and adults. It is thought to be the foundation for several disorders, e.g., obstructive sleep apnea, failure to thrive, abnormal chin and facial growth, difficulty swallowing (dysphagia), speech impairment, and chronic bad breath (halitosis). Chronic adenotonsillar hypertrophy and tonsillitis has been implicated in neurologic morbidity associated with sleep disorders, such as inattention and hyperactivity in children. Various other names have been used for or associated with the disease, such as “idiopathic adenotonsillar hypertrophy”, “obstructive adenotonsillar hypertrophy”, “chronic tonsillitis”, “acute recurrent tonsillitis”, “chronic adenotonsillitis”, “adenotonsillar hypertrophy”, or “obstructive sleep apnea syndrome (OSAS)”, especially in children.

Tonsils and adenoids are both secondary lymphatic organs that are located in the oropharynx and arranged in a circle around the pharynx, called Waldeyer's ring. Because of their similarity, the following discussion will be focused on the tonsils alone, unless otherwise specified. The tonsil contains four distinct compartments: 1) The oral squamous epithelial lining which extends down as 2) narrow epithelial channels, called crypts, deep into the underlying lymphoid tissue of the tonsil. Parallel to the crypt epithelium are 3) lymphoid follicles. There are two kinds of follicles: a) primary follicles which are composed of uniform compact spherical masses of small, non-dividing (resting) B lymphocytes, and b) secondary follicles which have a mantle zone of small non-dividing B cells surrounding a central mass of large, rapidly dividing B-lymphocytes, called the germinal centers (FIG. 1). Between the follicles is 4) the interfollicular area composed predominantly of T-lymphocytes. The germinal centers are a specialized microenvironment where B-lymphocytes undergo extensive proliferation, somatic mutation, antibody affinity maturation, and isotype switching, resulting in the formation of memory B cells and plasma cells. The tonsils increase in volume from birth to approximately 12 years of age and then involute during adolescence (Vaughn V C. Growth and development. In: Behrman R E and Vaughn V C. Editors, 1983. Nelson Textbook of Pediatrics Saunders, Philadelphia, Pa., pp. 10-38). This is a physiological process, reflecting the activity of the immune system in response to inhaled or swallowed antigens. The tonsils react to antigenic stimulation by increasing the number and size of germinal centers, leading to tonsillar enlargement or hypertrophy. These centers are more enlarged in primary than in secondary responses to antigens. Thus, tonsils enlarge in early life as a response to a large number of new antigens and involute in adolescence after the establishment of immune memory.

While most children do not have any pathological consequences associated with physical hypertrophy of the tonsils, some do develop recurrent acute tonsillitis or chronic adenotonsillar hypertrophy/tonsillitis (CATH/T). Recurrent acute tonsillitis requires tonsillectomy as definitive treatment when it exceeds seven episodes per year. Chronic adenotonsillar hypertrophy and tonsillitis can have a substantial impact on the quality of a child's life. A mild form of chronic adenotonsillar hypertrophy and tonsillitis, measured by sleep-associated gas exchange abnormalities (SAGEA), or using a more familiar term, snoring, is seen in 18.1% of children (Gozal Pediatrics (1998) 102: 616-620). SAGEA has an adverse effect on learning performance in elementary school. Significant improvement follows adenotonsillectomy. Children with lower academic performance in middle school are more likely to have snored during early childhood (Gozal et al., Pediatrics (2001) 1076:1394-1399), and this snoring-associated neuro-congnitive morbidity may be only partially reversible without therapeutic intervention. It is currently estimated that 20% to 30% of children with sleep disorders related to chronic adenotonsillar hypertrophy and tonsillitis may have clinically significant problems of inattention and hyperactivity due to repeated sleep arousals and intermittent hypoxia. Such children are in danger of being misclassified as having attention deficit hyperactivity disorder. Tonsillar hypertrophy also causes pharyngeal obstruction leading to mouth breathing, which can result in alterations in the development of muscular and bony facial structures in children (Subtelny, Ann Otol Rhinol Laryngol. (1975) 84(2 PT2 SUPPL 19): 50-54). The National Sleep Foundation estimated that 56% of men and 36% of women snore at least a few nights a week (Foley et al., J Psychosom Res. (2004) 56:497-502). Severe chronic adenotonsillar hypertrophy and tonsillitis can cause obstructive sleep apnea syndrome (OSAS), which occurs in approximately 1-3% of children (Ali et al., (1993) Arch. Dis. Child. 68: 360-366, Gislason et al., Chest (1995) 107:963-966; Redline et al., Am. J. Respir. Crit. Care. Med. (1999) 159:1527-1532) and 2-4% of adults (Young et al., N Engl J Med. (1993) 328:1230-5). Childhood OSAS was first described by William Osler (Osler, W. Chronic Tonsillitis. In: Osler, W. Editor, 1892. The Principles and Practice of Medicine Appleton, N.Y., pp. 335-339), but the first scientific case series was published only a few decades ago (Guilleminault et al., Pediatrics (1976) 58:23-30). Only then was it recognized that OSAS is a common disease, and that it can result in serious morbidity, including growth failure (Marcus et al., J. Pediatr. (1994) 125:556-562; Marcus et al., Obstructive sleep apnea syndrome. In: Loughlin, G. M. and Eigen, H. Editors, 1994. Respiratory Diseases in Children: Diagnosis and Management. Williams and Wilkins, Baltimore, pp. 475-499), cor pulmonale, and neuro-cognitive deficits (Gozal Pediatrics (1998) 102: 616-620), and even death has been reported (Marcus, supra).

Since its first histological observation in the tonsil in 1896, and its isolation in 1910 (Lord, J. Amer. Assoc. (1910) 55:1261-1263), Actinomyces species has become the most attractive candidate agent responsible for chronic adenotonsillar hypertrophy and tonsillitis. This has led to the widely held belief that ‘sulfur granule’ bacterial colonies, commonly seen in hypertrophic tonsils, are aggregates of Actinomyces species bacteria (FIG. 1). Indeed, in the literatures related to tonsillar diseases, “sulfur granules” and Actinomyces are synonymous. “Sulfur granules” can be as large as one millimeter in diameter. They are composed of tightly packed filamentous basophilic bacteria arranged in a radial spoke-like fashion (Pransky et al., Arch Otolaryngol Head Neck Surg. (1991) 117: 883-5). Several studies have reported various rates of histological detection of “sulfur granules” in recurrent acute tonsillitis (RAT) or chronic adenotonsillar hypertrophy and tonsillitis (CATH/T) ranging from 1.3% to 61.5%. The wide range of occurrence rates partially reflects historical differences in the classification of tonsillar diseases and the indications for tonsillectomy. In most cases, precise separation between RAT and CATH/T is impossible because of the coexistence of the two diseases. Bhargava et al. showed that there is a large difference in the detection rate of ‘Actinomyces’ between RAT and chronic tonsillar hypertrophy (CATH/T) (Bhargava et al., Acta Tropica (2001) 80:163-168). Maher et al cultured Actinomyces organisms from 30.4% of tonsils, but other studies failed to yield Actinomyces (Brodsky L. Ann Otol Rhinol Laryngol. (1991)100:1037; Van Staaij et al., Acta Otolaryngol. (2003) 123: 873-878; Maher et al., Laryngol Otol. (1982) 96:229-240). Chole et al. recently examined surgically-removed hypertrophic tonsils and demonstrated that the bacteria in ‘sulfur granule’-like macrocolonies are embedded in a homogeneous amorphous matrix consistent with a bacterial biofilm (Chole et al., Arch Otolaryngol Head Neck Surg. (2003)129:634-636). They state further that these bacteria are polymorphic, suggesting the granule is composed of multiple bacterial species rather than pure Actinomyces.

Several authors have attempted to cultivate pathogenic bacteria from CATH/T. The idea has been that CATH/T might be similar to acute tonsillitis in that a single pathogen is responsible for the disease. In acute tonsillitis, a significant percentage of cases are due to group A beta-hemolytic streptococci (GABHS), Epstein-Barr virus (EBV), or other upper respiratory pathogens, such as Hemophilus influenzae. However, in CATH/T, surface swabbing of tonsils as well as core biopsies yield mixed bacteria, often indistinguishable from oral bacterial biota, which is comprised of more than 600 bacterial species (Brodsky 1991, Van Staaij 2003, Reilly et al., J Clin Pathol. (1981) 34:542-547; Surow et al., Laryngoscope (1989) 99: 261-266; Gaffney et al., Clin Otolaryngol. (1993) 18:268-271; Gaffney et al., Respir Med. (1993) 87:303-308, Paster et al., J Bacteriol. (2001) 183:3770-83). Among mixed bacterial isolates, pathogens that are known to cause acute infections or carrier states include Hemophilus influenzae, Staphylococcus aureus, and Streptococcus pyogenes (Gaffney, supra, Surrow 1989, Ramirez et al., Enferm Infec Microbiol Clin. (1997) 15: 315-318), anaerobes include Fusobacterium sp Bacteroides sp, and Prevotella melaninogenica (Kuhn et al., Ann Otol Rhinol Laryngol. (1995) 104:646-652). However, there is no evidence that these bacteria are etiologically related to pathological lesions in hypertrophic tonsils.

Because of the lack of knowledge about the causative agent(s), there is no effective medical therapy for CATH/T. The vast majority of children with OSAS, however, improve after adenotonsillectomy (Suen et al., Arch. Otolaryngol. Head Neck Surg. (1995) 121:525-530). As a result, over 400,000 adenotonsillectomies are performed annually in the United States, making it one of the most common surgical procedures performed in children (National Center for Health Statistics 1996, Deustsch 1996, Derkay, Int J Pediatr Otorhinolaryngol. (1993) 25:1-12). The traditional adenotonsillectomy procedure is often a traumatic event, in which the tonsils and/or adenoids are cut out from their beds and capsule. The procedure is associated with a high level of pain and risk of bleeding in the post-operative period. Various surgical instruments have been provided in the prior art to reduce the pain and bleeding. For example, U.S. Pat. No. 6,682,501 teaches an apparatus and method for submucosal tonsillectomy without total removal of the tonsils. Another example, U.S. Pat. No. 6,530,924 teaches an electrosurgical electrode for use in a surgical procedure for a tonsillectomy or adenoidectomy procedure. These devices modify but do not change the invasive nature of the surgical treatment of chronic adenotonsillar hypertrophy and tonsillitis.

Sclafani et al (Sclafani et al., Pediatrics (1998) 101:675-681) empirically treated CATH/T with a 30-day course of amoxicillin/clavulanate potassium. The treatment was designed to target at Staphylococcus aureus, Streptococcus pneumoniae, Hemophilus influenza, and unspecified anaerobes because of the high incidence of these bacteria in diseased tonsils. The treatment caused a temporary relief in symptoms for reason unclear but the effect was associated with significant decreases in S. pneumoniae and H. influenza on the surface of the tonsils.

### SUMMARY OF THE INVENTION

In a first aspect, the present invention features an isolated Fusobacterium nucleatum corresponding essentially to the isolates Fusobacterium nucleatum ssp nucleatum (ATCC 25586) or Fusobacterium nucleatum spp vincentii (ATCC 49256) deposited with the American Type Culture Collection (ATCC), Manassas, Va. 20108 USA, and an isolated T. denticola essentially corresponds to the isolate (ATCC 35405)(Seshadri et al., Proc Natl Acad Sci USA. (2004) 101:5646-51), deposited with the American Type Culture Collection.

In a second aspect, the present invention features a method of diagnosing chronic adenotonsillar hypertrophy and tonsillitis (CATH/T) comprising detecting the presence of one or more bacteria selected from the group consisting of F. nucleatum and T. denticola in a biological sample. In some embodiments, the biological sample is blood or blood serum, sputum, saliva or urine. Detecting the bacteria may be performed by detecting the presence of a whole bacterial cell, an antigenic part or component of the bacteria, a protein or antigenic polypeptide derived from the bacteria, a peptide mimicking an antigenic component of the bacteria, antibodies in tonsil tissue specific for one or more antigens of the bacteria or lymphocytes having one or more surface receptors specific for one or more antigens of the bacteria. In some embodiments, the detecting is performed by an antibody detection assay such as ELISA or radioimmunoassay.

The antigens may be labeled with, for instance, with a fluorophore or a radioactive atom. Exemplary radioactive atoms include Technetium-99, Iodine-123, Iodine-131, Indium-111, Fluorine-19, Carbon-13, Nitrogen-15, Oxygen-17, Gadolinium, Manganese and Iron. In other embodiments, the detecting is performed by detecting the presence of one or more antigens of F. nucleatum or T. denticola in a biological sample. This may be performed, for instance, with an antibody specific for F. nucleatum or T. denticola. Assays well known to those skilled in the art such as sandwich ELISA or a radioimmunoassay may be used.

In other embodiments, the detecting may be performed by culturing samples obtained from the surface or crypts of the tonsils. In yet other embodiments, the detecting may be performed by detecting DNA of the bacteria in a biological sample such as tonsil tissue, saliva or blood. In still further embodiments, the detecting may be performed by detecting vaporizable compounds produced by the bacteria.

In a third aspect, the invention features a method for treating chronic adenotonsillar hypertrophy and tonsillitis comprising administering a pharmaceutically effective amount of an antibiotic capable of eradicating F. nucleatum and/or T. denticola. The treating may further involve disrupting granules in the tonsils. The antibiotic may be any one of several well known to those skilled in the art such as, for example, azithromycin, clindamycin, lincomycin, metronidazole, amoxicillin, clavulanate, cephalosporin, imipenem, and chloramphenicol. The antibiotic may be administered by any effective route such as sublingual, transmucosal, transdermal, parenteral, and oral. The antibiotic may be in some embodiments a radioactive antibody specific for F. nucleatum or T. denticola. Such a radioactive antibody may be conjugated to an agent such as Astatine-211, Iodine-131, Iodine-125, Yttrium-90, Rhenium-186, Rhenium-188, Samarium-153, Bismuth-212, Phosphorus-32, Lead-212 and Lutetium-177. Additionally, the treatment may include removing sulfur granules from hypertrophic tonsils.

In a fourth aspect, the present invention features a kit for diagnosing chronic adenotonsillar hypertrophy and tonsillitis comprising an agent capable of detecting the presence of F. nucleatum or T. denticola in a biological sample. The agent may be an antibody capable of detecting a F. nucleatum or T. denticola cell, a protein or polypeptide secreted by F. nucleatum or T. denticola or an antibody specific for F. nucleatum or T. denticola. In some embodiments, the agent may be an antibody labeled with a fluorophore or a radioactive atom.

In a fifth aspect, the invention features a vaccine for preventing chronic adenotonsillar hypertrophy and tonsillitis due to F. nucleatum and/or T. denticola comprising an agent capable of inducing an immune response to F. nucleatum or T. denticola. The agent capable of inducing an immune response to F. nucleatum or T. denticola may be an antigen of Fusobacterium or Treponema, a whole F. nucleatum or T. denticola cell, a protein or peptide secreted by F. nucleatum or T. denticola, or DNA encoding a protein or polypeptide produced by F. nucleatum or T. denticola. In instances where a whole bacterial cell is used, it is normally either killed or attenuated. The vaccine may further contain a suitable carrier or adjuvant.

### BRIEF DESCRIPTION OF THE FIGURES

FIG. 1 demonstrates the tonsillar architecture. The tonsil contains four distinct compartments: 1) The oral squamous epithelial lining which extends down as 2) narrow epithelial channels, called crypts, deep into the underlying lymphoid tissue of the tonsil. Parallel to the crypt epithelium are 3) lymphoid follicles. There are two kinds of follicles: a) primary follicles which are composed of uniform compact spherical masses of small, non-dividing (resting) B-lymphocytes, and b) secondary follicles which have a mantle zone of small non-dividing B cells surrounding a central mass of large, rapidly dividing B-lymphocytes, called the germinal centers. Between the follicles is 4) the interfollicular area composed predominantly of T-lymphocytes. In the figure, germinal centers (GC) and “sulfur granules” (SG) are shown in a tonsillar crypt.

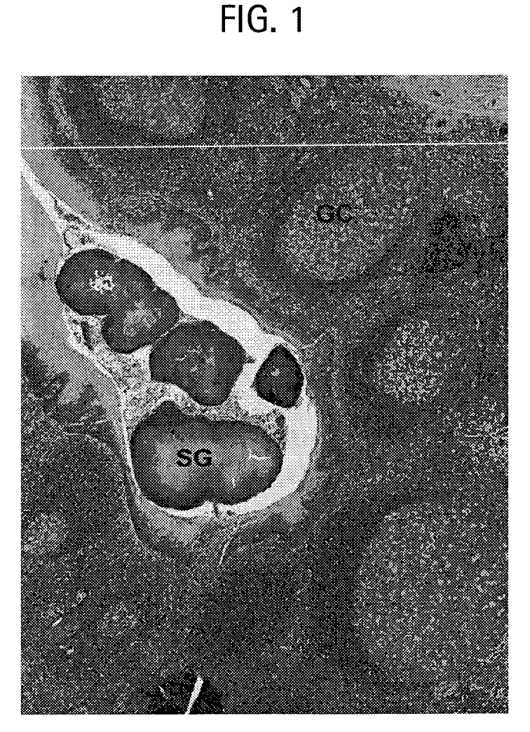

FIG. 2 demonstrates the “Sulfur granules” found in chronic tonsillitis and adenotonsillar hypertrophy. In the figure, a. is a gram stain of “sulfur granule”, and b. demonstrates a large and a small (upper right corner) “sulfur granule.”

FIG. 3 describes the results of broad-range 16S rDNA PCR, examining three cases. PCR products were cloned, yielding 276 clones: 43 from case one (T1), 37 from case two (T2), and 196 from case three (T3). To define a species boundary based on similarity scores used in the RDP II database (http://rdp.cme.msu.edu/), all 276 sequences from the three specimens were analyzed using linear regression. Each of the 276 sequences was paired with the best-matched sequences determined by Sequence Match. The similarity score and percentage of homology for each pair of sequences were calculated. The two measurements were related by the regression equation: y=0.0246x−1.493 (r=0.9122, p<0.0001), where y indicates the similarity score and x the percentage of sequence homology. Based on the regression formula, a similarity score of 0.891 was equivalent to the species boundary, defined by 97% 16S rDNA sequence homology (Stackebrandt et al., Int. J. Syst. Bacteriol. (1994) 44:846-849). All sequences with similarity scores <0.891 had <97% homology, and those >0.891 had homology ≧97%, without exception. The sequences defined by this method were termed species-level operational taxonomic units (SLOTU). This figure describes the results of fifty-five SLOTUs determined from 276 clones. None of the 276 clones were classified as being chimeric using the program “Chimera Detection” at RDP II. There were two species invariably present in all three “sulfur granules”: Fusobacterium nucleatum and Treponema denticola. F. nucleatum is the most prevalent species, accounting for 100 (36.2%) clones from the three “sulfur granules”. Further analysis revealed 3 subspecies of F. nucleatum in the “sulfur granules”, the most prevalent of which was F. nucleatum ssp vincentii. Fusobacterium AF287810 probably belongs to Fusobacterium nucleatum ssp nucleatum. Treponema denticola is the second most predominant species, accounting for 21 (7.6%) of the 276 clones. The frequency that a species was detected and its sources are indicated at right. The 55 SLOTU belonging to six phyla, contrasted by alternating red and blue print, are shown at right. SLOTU shared by all three persons are highlighted in yellow.

FIG. 4 demonstrates the results of ELISA measuring tonsillar IgG, IgA, and IgM reactions to the three bacterial species Fusobacterium nucleatum, Treponema denticola, and the negative control species Bacillus subtilis. Fusobacterium-specific IgA and IgG were present in 9 of 9 extracts and IgM in 4 of 9 extracts. Treponema-specific IgA, IgG, and IgM were positive in 4, 7, and 0 of the 9 extracts, respectively. Except for patient 1, all other 8 patients had significant level of antibodies to Treponema in at least one class of Ig.

### DETAILED DESCRIPTION

The present invention involves the discovery of the cause of chronic adenotonsillar hypertrophy and tonsillitis, previously an idiopathic disease, as being bacteria in “sulfur granules”, notably, Fusobacterium nucleatum and Treponema denticola. Based on this discovery, the current invention provides a diagnostic kit, diagnosis and treatment methods, and vaccines for chronic adenotonsillar hypertrophy and tonsillitis by the detection, treatment, and prevention of colonization/infection by F. nucleatum and/or T. denticola in tonsils.

The invention provides a composition comprising isolated Fusobacterium nucleatum and Treponema denticola, which are the causative agents of chronic adenotonsillar hypertrophy/tonsillitis. F. nucleatum essentially corresponds to the isolates Fusobacterium nucleatum ssp nucleatum (ATCC 25586) and Fusobacterium nucleatum spp vincentii (ATCC 49256) deposited with the American Type Culture Collection (ATCC), Manassas, Va. 20108 USA, and T. denticola essentially corresponds to the isolate (ATCC 35405)(Seshadri et al., Proc Natl Acad Sci USA. (2004) 101:5646-51), deposited with the American Type Culture Collection (ATCC), Manassas, Va. 20108 USA. The term “essentially corresponding” is intended to encompass variations that occur in nature and to artificial variations of F. nucleatum or T. denticola, including their subspecies, particularly those which still allow detection by techniques like hybridization, PCR and ELISA, using F. nucleatum-specific or T. denticola materials, such as F. nucleatum-specific or T. denticola-specific DNA or antibodies. Furthermore, since the entire genomes of F. nucleatum ssp nucleatum (Kapatral et al., J Bacteriol. (2002) 184:2005-2018), F. nucleatum ssp vincentii (Kapatral et al., Genome Res. (2003) 13:1180-1189) and T. denticola (Seshadri et al., Proc Natl Acad Sci USA. (2004) 101:5646-51) have been sequenced, their nucleotide sequences, genes, and proteins are all available for use in diagnostic kits and vaccines.

The present invention also provides methods for diagnosing chronic adenotonsillar hypertrophy and tonsillitis due to F. nucleatum and T. denticola. Previously, the diagnosis of chronic adenotonsillar hypertrophy and tonsillitis depended on direct visualization of the sizes of tonsils and adenoids but could not distinguish the physiological enlargement that may eventually regress from pathological enlargement due to the persistence of “sulfur granules” in the tonsils and adenoids. Since it is now known that chronic adenotonsillar hypertrophy and tonsillitis is caused by F. nucleatum and T. denticola, the said disease can be diagnosed by tests that detect the presence of the said bacteria in tonsils. Such diagnostic tests have until now not been available.

In one aspect, the invention provides methods for diagnosing chronic adenotonsillar hypertrophy and tonsillitis by detecting an antibody which specifically recognizes F. nucleatum or T. denticola or both in a sample. In particular, the biological sample may be blood or blood serum, sputum, saliva or urine. The method normally uses whole bacterial cells of F. nucleatum or T. denticola, or both, an antigenic part or component of F. nucleatum or T denticola, a protein or antigenic polypeptide derived from F. nucleatum or T. denticola, or a peptide mimicking an antigenic component of F. nucleatum or T. denticola. The method further utilizes a suitable detection means of an antibody detection assay. Alternatively, the tonsils or adenoids can be biopsied and antibodies can be extracted from the tissue and tested against antigens of Fusobacterium and/or Treponema. The presence of certain levels of antibodies to the bacteria could correlate with the presence of “sulfur granules” in the tonsils, therefore, indicating chronic adenotonsillar hypertrophy and tonsillitis due to F. nucleatum and T. denticola.

In another embodiment, the methods may detect chronic adenotonsillar hypertrophy and tonsillitis due to F. nucleatum and T. denticola by detecting the presence of lymphocytes that possess surface receptors to these bacteria, comprising fluorescent bacterial antigens of F. nucleatum and/or T. denticola. The fluorescent antigens may be labeled by one of various commercially available fluorophores or prepared by recombinant technology in which a gene encoding a protein of F. nucleatum or T. denticola or part of it is fused with a gene encoding a fluorescent protein or part of it, producing a fluorescent fusion protein. Lymphocytes may be prepared from the blood, tonsils, or adenoid. Lymphocytes recognizing F. nucleatum and/or T. denticola may be identified by contacting with the fluorescent bacterial antigens and scanning with a fluorescence microscope or flow cytometer. If a significant percentage of B cells recognize the antigens, a diagnosis of CATH/T due to F. nucleatum and/or T. denticola can be made.

Alternatively, the antigens may be labeled by a radioactive atom for scintigraphic examination of the presence of lymphocytes recognizing the antigens in the tonsils, for example Technetium-99 or Iodine-123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron. The labeled antigens may be injected intravenously, allowed to mix well with the blood, and diffuse into tonsils. The radioactivity is retained in the tonsils after contacting with lymphocytes that bind to the antigens. The tonsils are then imaged with a gamma-camera or NMR. Enlarged Tonsils not due to F. nucleatum and T. denticola will not take up the isotope and will appear as ‘cold’ on the tonsillar scan.

The radio- or other labels may be incorporated in the antigen in known ways. For example, the antigens may be biosynthesized or may be synthesized by chemical amino acid synthesis using suitable amino acid precursors involving, for example, fluorine-19 in place of hydrogen. Labels such as Technetium-99 or Iodine-123, rhenium-186, rhenium-188, and Indium-111 can be attached via a cysteine residue in the peptide. Yttrium-90 can be attached via a lysine residue. The IODOGEN method (Fraker et al., Biochem Biophys Res Commun. (1978) 80:849-57) can be used to incorporate iodine-123. “Monoclonal Antibodies in Immunoscintigraphy” (Chantal Monoclonal Antibodies in Immunoscintigraphy. CRC Press, 1989) describes other methods in detail.

In another embodiment, the method of diagnosing CATH/T is by detecting the antigens of F. nucleatum and/or T. denticola released into blood or tonsillar tissue or on to its surface, using antibodies to the antigens. The antibodies needed for these tests can be polyclonal or monoclonal antibodies directed against F. nucleatum and/or T. denticola. Methods to detect or quantify a specific antigen using antibodies are known in art. For instance, it is possible to use sandwich ELISA or radioimmunoassay to measure the presence of F. nucleatum and/or T. denticola in a biological sample from a patient with enlarged tonsils.

In yet another embodiment, the method of diagnosing chronic adenotonsillar hypertrophy and tonsillitis is by detecting the presence of F. nucleatum or T. denticola on the surfaces or the crypts of the tonsils to indicate their presence in the tonsillar crypt, by culturing the bacteria. Methods for culture and identification of F. nucleatum and T. denticola are known in art.

In yet another embodiment, the method of diagnosing chronic adenotonsillar hypertrophy and tonsillitis is by detecting the DNA of the bacteria present in tonsils or released into blood, by PCR or various DNA hybridization techniques, which are known in art. Since the sequences of the entire genomes of F. nucleatum and T. denticola are publicly available, specific nucleotide sequences can be identified and used to develop oligonucleotide sequences that can be used as probes or primers in diagnostic techniques such as hybridization, polymerase chain reaction, or any other techniques that are developed to specifically detect nucleotide sequences.

In yet another embodiment, the method of diagnosing chronic adenotonsillar hypertrophy and tonsillitis is by detecting vaporizable compounds produced by the bacteria, such as butyric acid, by gas, liquid, or gas-liquid chromatography, which are known in art.

In a second aspect, the current invention provides methods for treating chronic adenotonsillar hypertrophy and tonsillitis due to F. nucleatum and T. denticola by eradicating the bacteria. Previously, medical treatment of chronic adenotonsillar hypertrophy and tonsillitis with antibiotics was inefficient. This could be due to the poor selection of antibiotics because the target bacteria were unknown. With the knowledge of causative bacteria of chronic adenotonsillar hypertrophy and tonsillitis, specific treatment regimens can be designed to target the bacteria. Alternatively, chemical or physical treatment can be designed to disrupt the granules, thus making the bacteria in the granules more accessible to antibiotics or the human immune system.

In a third aspect, the invention provides methods for treating chronic adenotonsillar hypertrophy and tonsillitis by using antibiotics targeting F. nucleatum or T. denticola, or both, in tonsils. In accordance with the invention, the antibiotic therapy may be comprised of one or more of the following classes of antibiotics: azithromycin, clindamycin, lincomycin, metronidazole, amoxicillin, clavulanate, cephalosporin, imipenem, chloramphenicol, and tetracycline. The antibiotic products may be given at dosages according to those used in medical practice for adults and children. The antibiotic composition may be administered for example, by any one of the following routes of administration: sublingual, transmucosal, transdermal, parenteral, oral, preferably by oral administration.

The invention further provides methods for treating chronic adenotonsillar hypertrophy and tonsillitis by disruption of “sulfur granules” in the tonsils using antibodies targeting F. nucleatum and/or T. denticola in tonsils. For selective destruction of the “sulfur granules”, the antibody against F. nucleatum or T. denticola may be hooked to a highly radioactive atom. A variety of radioactive isotopes are available for the production of radioconjugated anti-F. nucleatum or T. denticola antibodies. Examples include astatine-211, Iodine-131, Iodine-125, Yttrium-90, Rhenium-186, Rhenium-188, Samarium-153, Bismuth-212, Phosphorus-32, Lead-212 and radioactive isotopes of Lutetium-177. The radioconjugated antibodies may be delivered to the hypertrophic tonsils by injection into the tonsillar crypts or parenteraly to allow their concentration in the tonsils through binding to F. nucleatum and/or T. denticola of the “sulfur granules”.

The invention further provides methods for treating chronic adenotonsillar hypertrophy and tonsillitis by physically removing “sulfur granules” from the hypertrophic tonsils. U.S. Pat. No. 6,475,172 (Hall 2000) describes a tonsil cleansing tool for cleaning debris and build-up from a tonsillar pit or cavity of a tonsil thereby eliminating bad breath. The current invention will provide a similar tool for treating chronic adenotonsillar hypertrophy and tonsillitis. The tool may apply pressure to the openings of the crypts and push out the “sulfur granules”. Alternatively, a cleaning tool may apply a vacuum to the surface of the tonsil and suck out the “sulfur granules” from the crypts.

The current invention further provides vaccines and compositions for prevention of chronic adenotonsillar hypertrophy and tonsillitis due to F. nucleatum and T. denticola. Because of the lack of knowledge of the cause of chronic adenotonsillar hypertrophy and tonsillitis, there have been no measures to prevent the disease. With the knowledge of the causative bacteria disclosed through this invention, specific vaccination for children with antigens of Fusobacterium and/or Treponema, whole bacterial cells, their subcellular components, proteins, peptides or DNA encoding their proteins could be protective.

In a fourth aspect, the present invention features a kit for diagnosing chronic adenotonsillar hypertrophy and tonsillitis comprising an agent capable of detecting the presence of F. nucleatum or T. denticola in a biological sample. The agent may be an antibody capable of detecting a F. nucleatum or T. denticola cell, a protein or polypeptide secreted by F. nucleatum or T. denticola or an antibody specific for F. nucleatum or T. denticola. In some embodiments, the agent may be an antibody labeled with a fluorophore or a radioactive atom. Kits may be developed that contain the antibodies, reagents, buffers, standards and instructions for assaying both enzymes using the same format, e.g. ELISA, or a colorimetric assay. The test kits would be modified appropriately depending on whether the samples to be assayed consist of whole cells, cell lysates or a combination thereof.

In a fifth aspect, the invention provides a vaccine composition for humans, to protect them against chronic adenotonsillar hypertrophy and tonsillitis, comprising F. nucleatum or T. denticola, or both, either killed with formalin or ultra-violet light or attenuated. In another embodiment, the vaccine may comprise a recombinant vector which contains a nucleotide sequence coding for a protein or antigenic peptide derived from F. nucleatum or T. denticola, an antigenic part or component of F. nucleatum or T. denticola, a protein or antigenic polypeptide derived from, or a peptide mimicking an antigenic component of F. nucleatum or T. denticola. The vaccine may further include a suitable carrier or adjuvant such as aluminum hydroxide or others. This composition may then be delivered by any suitable route of administration such as sublingual, transmucosal, transdermal, parenteral, oral, or intranasal.

Live vaccines against chronic adenotonsillar hypertrophy and tonsillitis can be made by recombinant DNA techniques through which the gene(s) encoding antigens of F. nucleatum, or T. denticola, is incorporated in vector systems such as vaccinia virus, herpesvirus, pseudorabies virus, adenovirus or other suitable vector systems that can so express the antigen. Humans, vaccinated with such live vector systems would then develop protective immune responses and be protected from developing chronic adenotonsillar hypertrophy and tonsillitis.

Fusobacterium is notorious for production of malodor. The current invention indicates that it is a source of malodor from chronic adenotonsillar hypertrophy and tonsillitis tonsils. Therefore, the diagnostic kits, treatment methods except for the physical method, and vaccines also apply to malodor or bad breath (halitosis) related to chronic adenotonsillar hypertrophy and tonsillitis.

It is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the particular methods and materials are now described. All publications mentioned herein are incorporated herein by reference.

Definitions

The terms used herein have the meanings recognized and known to those of skill in the art; however, for convenience and completeness, particular terms and their meanings are set forth below.

“Treatment” refers to the administration of a drug or the performance of procedures with respect to a subject, for either prophylaxis (prevention) or to cure the infirmity or malady in the instance where the subject is afflicted.

By “individual” or “patient” or “subject” is meant a human or non-human mammal that may benefit from the diagnostic tests or methods described in the present application, for example, an individual at risk for developing or having chronic adenotonsillar hypertrophy.

Assay Formats

The methods of this invention may use assays, which may be practiced, in almost a limitless variety of formats depending on the particular needs at hand. Such formats include, but are not limited to traditional “wet chemistry” (e.g. as might be performed in a research laboratory), high-throughput assay formats (e.g. as might be performed in a pathology or other clinical laboratory), and “test strip” formats, (e.g. as might be performed at home or in a doctor's office).

Traditional Wet Chemistry

The assays of this invention can be performed using traditional “wet chemistry” approaches. Basically this involves performing the assays as they would be performed in a research laboratory. Typically the assays are run by incubation of biological samples on a culture media or with immobilized antibodies or antigens.

High-throughput Assay Formats

Where population studies are being performed, and/or in clinical/commercial laboratories where tens, hundreds or even thousands of samples are being processed (sometimes in a single day) it is often preferably to perform the assays using high-throughput formats. High throughput assay modalities are highly instrumented assays that minimize human intervention in sample processing, running of the assay, acquiring assay data, and (often) analyzing results. In particular embodiments, high throughput systems are designed as continuous “flow-through” systems, and/or as highly parallel systems.

Flow through systems typically provide a continuous fluid path with various reagents/operations localized at different locations along the path. Thus, for example a blood sample may be applied to a sample receiving area where it is mixed with a buffer, the path may then lead to a cell sorter that removes large particulate matter (e.g. cells), the resulting fluid may then flow past various reagents (e.g. where the reagents are added at “input stations” or are simply affixed to the wall of the channel through which the fluid flows.

In highly parallel high throughput systems samples are typically processed in microtiter plate formats (e.g. 96 well plates, 1536 well plates, etc.) with computer-controlled robotics regulating sample processing reagent handling and data acquisition. In such assays, the various reagents may all be provided in solution. Alternatively some or all of the reagents may be provided affixed to the walls of the microtiter plates.

High throughput screening systems that can be readily adapted to the assays of this invention are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols.

“Test Strip” Assay Formats

The methods of the present invention may also utilize assays which are provided in “test well” or “test strip” formats. In “test well” or “test strip” formats, the biological sample is typically placed in the well or applied to a receiving zone on the strip and then a fluorescent or calorimetric indicator appears which, in this case, provides a measure of the level of the agent.

In some embodiments, an assay format is provided in which binding partners such as antibodies can be obtained or prepared for F. nucleatum or T. denticola. Biotin-avidin, biotin-streptavidin or other biotin-binding-reagent reactions can be used to enhance or modulate the test. However, any such assay can be devised using other binding partners to the analytes, including but not limited to extracellular or intracellular receptor proteins which recognize the analytes, binding fragments thereof, hybridization probes for nucleic acids, lectins for carbohydrates, etc. The particular selection of binding partners is not limiting, provided that the binding partners permit the test to operate as described herein. The preselected analytes, when present, are detectable by binding two binding partners, one immobilized on the test strip (or whatever format the assay is provided) and another part of a conjugate. This is taken into consideration in the selection of the reagents for the assay.

If a dry test strip is desired, this may be set up in any format in which contact of the sample with the reagents is permitted and the formation and mobility of the immunocomplexes and other complexes forming therein are permitted to flow and contact an immobilized reagent at the capture line. Various formats are available to achieve this purpose, which may be selected by the skilled artisan.

The label portion of the mobile, labeled antibody to the marker may be a visible label, such as gold or latex, an ultraviolet absorptive marker, fluorescent marker, radionuclide or radioisotope-containing marker, an enzymatic marker, or any other detectable label. A visibly detectable marker or one that can be easily read in a reflectometer is preferred, for use by eye, reading or confirmation with a reflectometer. Other labels may be applicable to other semi-automated or automated instrumentation.

The conjugates of the invention may be prepared by conventional methods, such as by activation of an active moiety, use of homobifunctional or heterobifunctional cross-linking reagents, carbodiimides, and others known in the art. Preparation of, for example, a gold-labeled antibody, a conjugate between an antibody and an analyte (not an immunocomplex but a covalent attachment which allows each member to independently exhibit its binding properties), biotinylation of an antibody, conjugation of streptavidin with a protein, immobilization of antibodies on membrane surfaces, etc., are all methods known to one of skill in the art.

### DETAILED DESCRIPTION OF THE PREFERRED EMBODIMENTS

### Example 1

A large percentage (1.3-61%) of CATH/T tonsils contain microscopically visible clusters of bacteria called “sulfur granules” or “Actinomyces granules”, but it was not known whether they are also present in normal tonsillar tissues.

Experimental Design and Methods:

We studied consecutive patients who underwent tonsillectomy. The inclusion criteria were patients with a clinical diagnosis of chronic tonsillitis, recurrent tonsillitis, tonsillar hypertrophy, or a combination of these diagnoses. Cases were excluded if there were recent episodes of purulent acute tonsillitis or if the tonsils were fragmented into multiple pieces, preventing accurate measurement of the dimensions of the tonsils for the estimation of tonsillar hypertrophy. Of the 290 tonsillectomy cases due to inflammatory tonsillar diseases, 16 were excluded, 5 due to fragmentation of the specimen and 11 due to recent peritonsillar abscess. Clinically, 248 patients were diagnosed as chronic tonsillitis, 2 as recurrent tonsillitis, 5 as tonsillar hypertrophy, and 19 as a combination of these diagnoses. Because clinical diagnosis often was vague and incomplete, we defined tonsillar hypertrophy as meeting the following three objective criteria: 1) the mean greatest dimension of the pair of tonsils was greater than 50% of the maximum size at puberty (Williams 1995) [Tonsils change size with age and inflammation, therefore, it is difficult to define normal size. At puberty, the tonsil reaches its maximum size, averaging 2.0 to 2.5 cm in the greatest dimension. Tonsillar involution begins at puberty and by old age only a little tonsillar lymphoid tissue remains. Considering the age factor and the inconstant nature of the reference value, we chose 50% of the maximum as the cutoff value (1.0-1.25) for tonsils to be considered as hypertrophic], 2) the presence of histological evidence of secondary follicles accounting for >50% of all lymphoid follicles, and 3) the absence of histological evidence of tissue necrosis, a hallmark for acute suppurative inflammation. In the 274 patients, the tonsils were 2.7±0.52 cm in greatest mean dimension (range 1.3-3.75 cm), all being significantly larger that the cutoff threshold. Microscopic examination further excluded 11 other cases, 6 due to the lack of significant lymphoid hyperplasia and 5 due to tissue necrosis. The remaining 263 cases were categorized as CATH/T based on the chronic history of tonsillar disease and histologic evidence of hyperplasia in the absence of acute tonsillitis.

Results:

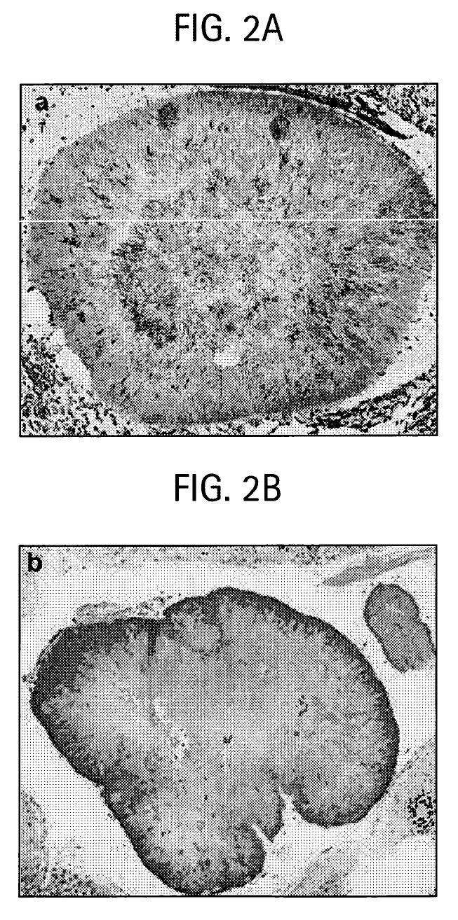

“Sulfur granules” (FIG. 2) were identified in 111 (42.2%) of the 263 patients.

One hundred non-inflammatory tonsillar cases were available for use as controls, consisting of 1 case of tonsillar cyst, one case of tonsillar scar, and 98 cases of tonsillar biopsies for diagnosis of carcinoma. Both the tonsillar cyst and scar specimens were negative for the presence of “sulfur granules”. Of the 98 cases of tonsillar biopsies, 42 were excluded due to the lack of tonsillar lymphoid tissue in the specimens. Of the remaining 56 cases of tonsillar biopsies, two cases contained “sulfur granules” (3.4%).

Conclusion:

The difference between the detection rate of “sulfur granules” in CATH/T and in non-inflammatory controls is very significantly different (42.2% vs 3.4%, P<0.001), indicating that there is a strong association of “sulfur granules” and CATH/T.

### Example 2

### Detection Rate of “Sulfur Granules” in Adequately Sampled CATH/T Tonsils

The detection rate of “sulfur granules” may vary with the extent to which the tonsils were sampled. In a surgical pathology laboratory, it is routine to examine one section for each tonsil, as is evident in the archival material we examined.

Methods and Results:

Two tonsils were examined for each patient with CATH/T. Fresh tonsils were sectioned every 2 to 3 mm and all sections were processed for histological examination. Of the 3 cases examined, only case 1 would have been found to contain “sulfur granules” if only two sections were examined. However, when adequately sampled, “sulfur granules” were observed in all 3 cases examined, suggesting that nearly all CATH/T tonsils contain “sulfur granules” and that all CATH/T cases may have the same etiology (Table 1).

TABLE 1
Detection of “sulfur granules” in adequately sampled CATH/T tonsils

| | Number | Number of sections | | False |
|---|---|---|---|---|
| | of | positive | Sensitivity | negatively |
| Case | sections | for “sulfur granules” | per 2 sections | per 2 sections |
| 1 | 14 | 10 | 92% | 8% |
| 2 | 17 | 2 | 22% | 78% |
| 3 | 18 | 1 | 11% | 89% |
| 4 | 10 | 3 | 51% | 49% |
| 5 | 11 | 1 | 17% | 83% |
| 6 | 17 | 3 | 32% | 68% |
| 7 | 16 | 12 | 94% | 6% |
| Average | 14.7 | 4.6 | 52% | 48% |

### Example 3

### Defining the Bacterial Species Composition of “Sulfur Granules” using 16S PCR-based Sequencing

The candidate antigens responsible for the B lymphocyte activation and hyperplasia are likely to be from bacteria residing in the “sulfur granules”. However, no bacterial species in the granules has been phylogenetically identified until the present study.

Methods:

Using universal 16S rDNA PCR, biopsies were examined from the tonsils of three cases of CATH/T. “Sulfur granules” were dissected from sections of formalin-fixed tonsils in two cases (T1 and T2) using microscopy and isolated from a fresh tonsil in one case (T3) under a stereomicroscope.

DNA was extracted from the “sulfur granules” using a tissue DNA extraction kit (Qiagen). To lyse bacteria, the isolated “sulfur granule” was incubated with lysozome (20 mg/ml) (Sigma) in 180 μl of buffer containing 20 mM Tris HCl, pH 8.0, 2 mM EDTA, and 1.2% Triton X-100 for 60 min at 37° C. The Qiagen tissue DNA extraction protocol was then followed and the DNA-enriched fractions were eluted in 200 μl of H₂O.

Bacterial 16S rRNA genes (rDNA) in the extracted DNA were analyzed by universal bacterial 16S PCR. Five μl of the extracted DNA from each “sulfur granule” were used as templates in a 50 μl PCR reaction. PCR assays were conducted in a thermocycler under the following conditions: 2 min at 94° C., 30 cycles with each cycle consisting of 30 seconds at 94° C., 30 seconds at 60° C., and 30 seconds at 72° C. A 72° C. extension was added at the end of the 30th cycle. For the “sulfur granule” isolated from the fresh tonsil, primers 8f and 1510r were used. Primers 8f and 1510r have been widely used (Paster et al., J Bacteriol. (2001) 183:3770-83; Pei et al., Proc Natl Acad Sci USA. (2004) 101:4250-5) and have exhibited no significant amplification bias. For the “sulfur granules” dissected from the fixed tonsils, PCR reactions were performed with primers 21F: 5′-TGCAIGGIIGTCGTCAGCTC-3′ and 27R: 5′-ACGTCITCCICICCTTCCTC-3′, where I represents inosine, which was used at positions of nucleotide ambiguity, since it forms stable base pairs with all four usual bases. The amplification products were separated from free primers and/or primer-dimers using 1.0% agarose gel electrophoreses. The portion of gel containing the PCR products was excised and the DNA was purified using a Gel extraction kit (Qiagen). The purified PCR product was ligated into pGEM T easy cloning vector and then used to transform competent E. coli DH5α cells. After transformation, the E. coli cells were plated onto LB agar plates in the presence of ampicillin. The cloned 16S rRNA gene was re-amplified with the primer 8f and 1510r. Free primers were removed from the PCR products using Qiagen PCR cleaning kit. The purified PCR products were then sequenced in a commercial DNA sequencing laboratory. Sequences were aligned using Clustal X and vector and primer sequences were edited.

To assign PCR clones to the accurate taxon, species level operational taxonomic unit (SLOTU) was first defined. This is necessary because RDP II (RDPII, http://rdp.cme.msu.edu) Sequence Match (Preview Version) compares the inquiry sequence to known 16S rDNA sequences for assignment to the closest taxon based on similarity scores. In the literature, 97% sequence homology of 16S rDNA has been defined as the species boundary (Stackebrandt et al., Int. J. Syst. Bacteriol. (1994) 44:846-849). However, its mathematical relation with the RDP II similarity score had to be determined based on the set of sequences to be analyzed. We defined the cut-off value for the SLOTU in this study using the method previously described in analysis of esophageal bacterial biota (Pei et al., Proc Natl Acad Sci USA. (2004) 101:4250-5). For phylogenetic treeing analysis, representative 16S rDNA sequences were aligned using Sequence Aligner at RDP II. Misaligned positions were corrected using ARB (http://rtfm.arb-home.de). Phylograms of the nucleotide alignment were generated using the PAUP 4.0b10 (PAUP 4.0b2. Phylogenetic Analysis Using Parsimony Version 4, Sinauer Associates, Sunderland, Mass.) and the neighbor-joining method. Robustness of the resulting phylograms was determined using bootstrapping (PAUP).

The total number of species-level operational taxonomic units (SLOTU) that may be present in “sulfur granules” and its associated confidence interval were calculated using a nonparametric richness estimator, Chao 1, as described (Chao, Biometrics (1987) 43:783-791).

Results Defining Species-Level Operational Taxonomic Units (SLOTU):

Using broad-range 16S rDNA PCR, biopsies were examined from three cases. PCR products were cloned, yielding 276 clones (FIG. 5): 43 from case one (T1), 37 from case two (T2), and 196 from case three (T3). To define a species boundary based on similarity scores used in the RDP II database, all 276 sequences from the two specimens were analyzed using linear regression. Each of the 276 sequences was paired with the best-matched sequences determined by Sequence Match. The similarity score and percentage of homology for each pair of sequences were calculated. The three measurements were related by the regression equation: y=0.0246x−1.493 (r=0.9122, p<0.0001), where y indicates the similarity score and x the percentage of sequence homology. Based on the regression formula, a similarity score of 0.891 was equivalent to the species boundary, defined by 97% 16S sequence homology (Stackebrandt et al., Int. J. Syst. Bacteriol. (1994) 44:846-849). All sequences with similarity scores <0.891 had <97% homology, and those >0.891 had homology ≧97%, without exception. The sequences defined by this method were termed Species-Level Operational Taxonomic Units (SLOTU).

Elimination of Contaminating Sequences:

The PCR reagent control, including all PCR reagents except for the template, did not generate any visible signals. The DNA extraction control, using water to replace the “sulfur granules” during the extraction, generated a faint band. The band was excised, the PCR products cloned, and sequence determined. Eleven phylotypes were found relating to the genera Pseudomonas, Acinetobacter, Streptococcus, Staphylococcus, Delftia acidovorans, Brochotrix thermosphacta, Flavobacterium, Rickettsia, and Janthinobacterium. None of these sequences were found in the PCR clones from the “sulfur granules”.

Determination of the Species Composition of “Sulfur Granules”:

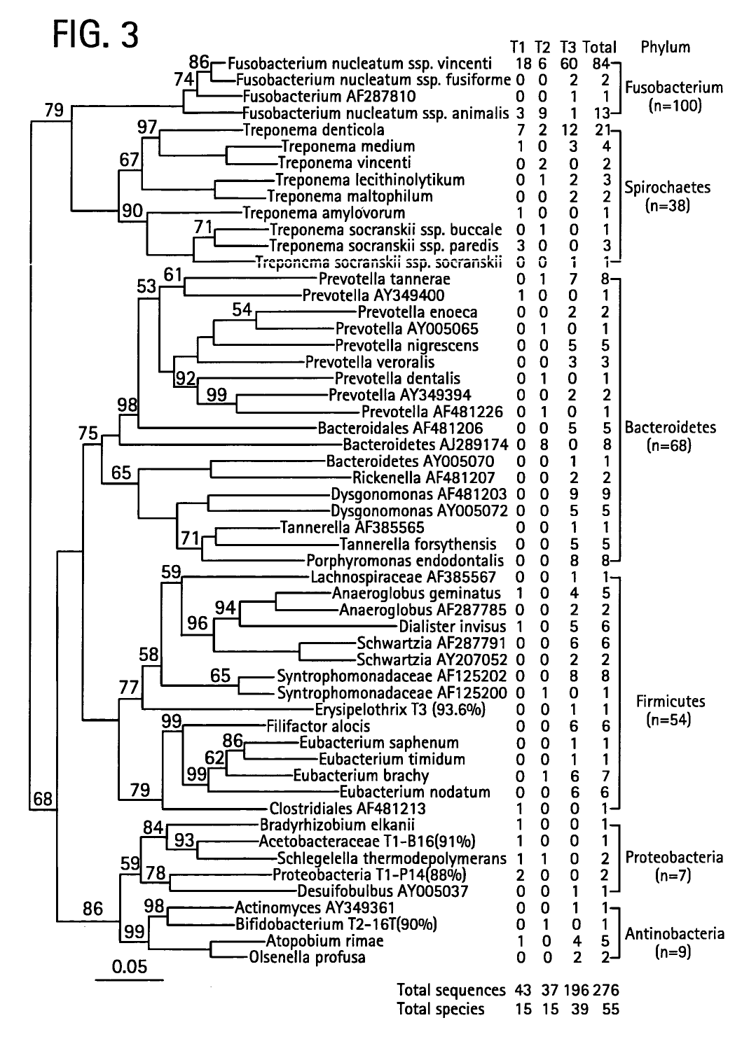

Fifty-five SLOTUs were determined from the 276 clones (FIG. 3). None of the 276 clones were classified as being chimeric using the program “Chimera Detection at RDP II”. There were two species invariably present in all three “sulfur granules”: Fusobacterium nucleatum and Treponema denticola. F. nucleatum is the most prevalent species, accounting for 100 (36.2%) clones from the three “sulfur granules”. Further analysis revealed 3 subspecies of F. nucleatum in the “sulfur granules” (FIG. 3), most prevalent of which were F. nucleatum ssp vincentii. Fusobacterium AF287810 probably belongs to Fusobacterium nucleatum ssp nucleatum. Treponema denticola is the second most predominant species, accounting for 21 (7.6%) of the 276 clones. Three genera, Fusobacterium, Treponema, and Prevotella were represented in 3 of the 3 “sulfur granules”. In total, members of the three genera comprised 162 (58.7%) of the 276 clones sampled from the sulfur granules”. The other taxonomic groups were only sporadically found in some species. Surprisingly, only one clone, Actinomyces AY349361, was found to belong to Actinomyces.

Estimation of SLOTU Richness:

Chao 1 estimation (Chao 1987) projected a composition of 96 SLOTU (95% Confidence Interval, 78-121%) within the “sulfur granules”. Based on this projection, this study has identified ˜57.3% (95% Confidence Interval, 45.5 to 70.5%) of the SLOTU in this bacterial ecosystem.

Conclusions:

Ninety six SLOTUs were projected to comprise the “sulfur granules”, an estimation based on the 276 clones examined. This study has identified 55 SLOTU or ˜57.3% (95% Confidence Interval, 45.5 to 70.5%) of the total SLOTU potentially present in this bacterial ecosystem. Two bacterial species were invariably present in all three “sulfur granules” examined: Fusobacterium nucleatum (36.2%) and Treponema denticola (7.6%). Besides these, the “sulfur granules” from each case also contained minor bacteria that were not common among cases. Assuming that there is a common basis for the etiology of CATH/T, these non-common members are less likely to significantly contribute to the activation of B-lymphocytes in CATH/T.

### Example 4

### Response and Specificity of Local Tonsillar Antibodies to “Sulfur Granule”-common Species

Demonstration of an etiologic relationship requires specific links between the agent(s) and the disease beyond simple associations. Because antibodies in tonsils could be the end products of B-lymphocytes in CATH/T, which may reflect the specificity to the original stimulating antigens, study of tonsillar antibodies could provide insight into the specificity of membrane-bound antibodies on B-lymphocytes and could elucidate antigens responsible for B-lymphocyte activation in CATH/T.

Methods:

One-half gram of fresh tonsil from each tonsillectomy specimen of the 9 patients with CATH/T was minced into 1 mm or smaller pieces. The minced tissue was then washed with phosphate buffered saline (PBS) twice to reduce blood contamination and then grounded to homogeneity in 1 ml of PBS. After centrifugation, the supernatant represented the tonsillar antibodies, and was used at 1:20 dilution. Whole cell antigens of F. nucleatum ssp nucleatum and T. denticola were used in ELISA to determine the specificity of the tonsillar antibodies. Bacillus subtilis, a soil bacterium rarely seen in humans, served as a negative control.

To sensitize the ELISA plates (Immulon II; Dynatech Laboratories Inc., Alexandria, Va.), bacterial antigens were diluted to 10 μl/ml in 0.015 M carbonate buffer, pH 9.6. One hundred μl of the antigens was added to each well and the plates were incubated at 4° C. overnight. The plates were washed once with 0.01 M PBS, pH 7.2, in 0.03% Tween 20 and 0.01% thimerosal (PBS-T-T) and then blocked with 200 μl per well of 0.1% gelatin in PBS-T-T overnight at 4° C. After washing, 100 μl of tonsillar antibodies, diluted to 1:20 with 0.5% bovine y-globulin and 0.1% gelatin in PBS-T-T, was added in triplicate to each well. After 1 h incubation at 37° C., the plates were washed three times as before and 100 μl diluted peroxidase-rabbit anti-human IgG, IgM, or IgA was added to each well and incubated at 37° C. for 1 h. After washing 5 times, 100 μl of peroxidase developer (20 mg of 2.2′-azinobis (3-ethylbenzothiazoline-6-sulfonic acid), 34 μl of H₂O₂, 9.35 ml of 0.2 M Na₂HPO₄, and 10.65 ml of 0.1 M citric acid) were added to each well. The product of the peroxidase reaction was determined after 30 min at room temperature using a MR 600 microplate reader (Dynatech Laboratories Inc., Alexandria, Va.) at 414 nm. Tonsillar IgG, IgA, and IgM reactions to the antigens were measured. The cutoff values were determined by the mean optical +3 Standard Deviation (SD) of the antibody reactions in comparison to the control bacterium, Bacillus subtilis.

Results:

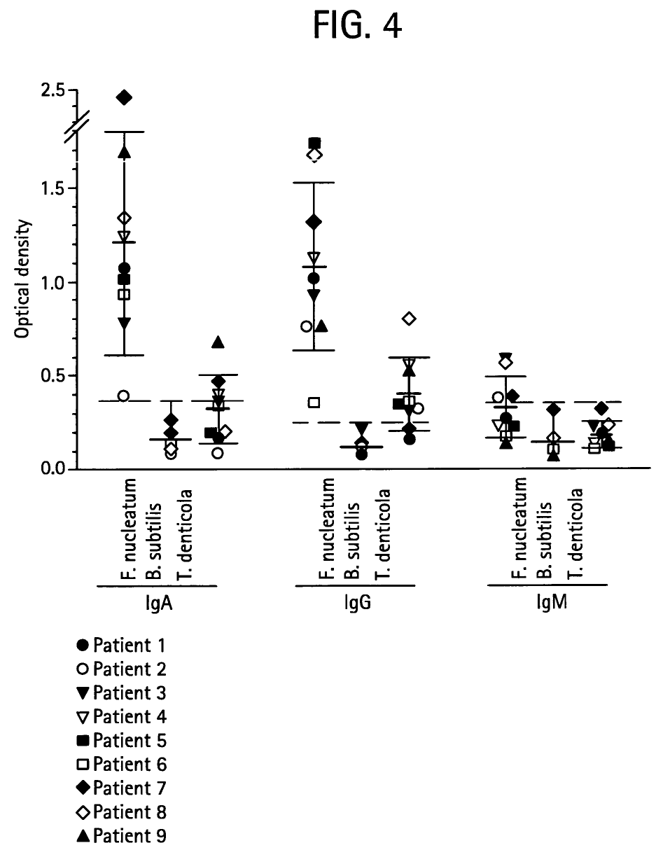

ELISA measured the tonsillar IgG, IgA, and IgM reactions to the three bacterial species Fusobacterium nucleatum, Treponema denticola, and Bacillus subtilis. Fusobacterium-specific IgA and IgG were present in 9 of 9 extracts and IgM in 4 of 9 extracts (FIG. 4). The predominant type of antibody varied among patients. For example, while patient 2 displayed borderline positivity to IgA and IgM and a much stronger IgG reaction to Fusobacterium, patient 6 was negative to IgM, weakly positive to IgG, but showed strong IgA positivity. Treponema-specific IgA was positive in 4 extracts, IgG in 7 extracts, and IgM in none. Except for patient 1, all other 8 patients had a significant level of antibody response to Treponema in at least one class of Ig. None of the patients exhibited a significant antibody reaction to Bacillus in any class of immunoglobulin.

Conclusion:

We examined tonsillar antibodies from 9 patients with CATH/T and found that nearly all CATH/T tonsils contain antibodies specific to antigens prepared from both Fusobacterium and Treponema. High concentrations of antibodies to F. nucleatum were observed in 9 of 9 cases examined and a low but significant concentration was observed in antibodies to T. denticola in 8 of the 9 cases examined. These results show that there is a specific, etiologic link between F. nucleatum and T. denticola and chronic adenotonsillar hypertrophy.

What is claimed is:

1. An isolated Fusobacterium nucleatum corresponding to the isolates Fusobacterium nucleatum ssp nucleatum (ATCC 25586) or Fusobacterium nucleatum spp vincentii (ATCC 49256) deposited with the American Type Culture Collection (ATCC).

2. A method of diagnosing chronic adenotonsillar hypertrophy and tonsillitis comprising detecting the presence of one or more bacteria selected from the group consisting of F. nucleatum and T. denticola in a biological sample.

3. A method according to claim 3 wherein the biological sample is selected from the group consisting of blood or blood serum, sputum, saliva, urine.

4. A method according to claim 3 wherein the detecting is performed by detecting the presence of a whole bacterial cell.

5. A method according to claim 3 wherein the detecting is performed by detecting the presence of an antigenic part or component of the bacteria.

6. A method according to claim 3 wherein the detecting is performed by detecting the presence of a protein or antigenic polypeptide derived from the bacteria.

7. A method according to claim 3 wherein the detecting is performed by detecting the presence of a peptide mimicking an antigenic component of the bacteria.

8. A method according to any of claims 3-7 wherein the detecting is performed by an antibody detection assay.

9. A method according to claim 3 wherein the detecting is performed by determining the presence of antibodies in tonsil tissue specific for one or more antigens of a bacteria selected from the group consisting of Fusobacterium and Treponema.

10. A method according to claim 3 wherein the detecting is performed by detecting the presence of lymphocytes having one or more surface receptors specific for one or more antigens of a bacteria selected from the group consisting of F. nucleatum and T. denticola.

11. A method according to claim 10 wherein the antigens are labeled.

12. A method according to claim 11 wherein the antigens are labeled with a fluorophore.

13. A method according to claim 12 wherein the fluorophore is encoded by a gene encoding a fluorescent protein or part of it fused to a gene encoding a protein of F. nucleatum or T. denticola thereby producing a fluorescent fusion protein.

14. A method according to claim 11 wherein the antigens are labeled with a radioactive atom.

15. A method according to claim 14 wherein the radioactive atom is selected from the group consisting of Technetium-99, Iodine-123, Iodine-131, Indium-111, Fluorine-19, Carbon-13, Nitrogen-15, Oxygen-17, Gadolinium, Manganese and Iron.

16. A method according to claim 3 wherein the detecting is performed by detecting the presence of one or more antigens of F. nucleatum or T. denticola in a biological sample.

17. A method according to claim 16 wherein the biological sample is selected from the group consisting of blood, saliva and tonsil tissue.

18. A method according to claim 16 wherein the antigen is detected by an antibody specific for F. nucleatum or T. denticola.

19. A method according to claim 16 wherein the detecting is performed by an assay selected from the group consisting of a sandwich ELISA or a radioimmunoassay.

20. A method according to claim 3 wherein the detecting is performed by culturing the surface or crypts of the tonsils.

21. A method according to claim 3 wherein the detecting is performed by detecting DNA of the bacteria in tonsils, saliva or blood.

22. A method according to claim 3 wherein the detecting is performed by detecting vaporizable compounds produced by the bacteria.

23. A method for treating chronic adenotonsillar hypertrophy and tonsillitis comprising administering a pharmaceutically effective amount of an antibiotic capable of eradicating F. nucleatum or T. denticola.

24. A method according to claim 23 further comprising disrupting granules in the tonsils.

25. A method according to claim 23 wherein the antibiotic is selected from the group consisting of azithromycin, clindamycin, lincomycin, metronidazole, amoxicillin, clavulanate, cephalosporin, imipenem, and chloramphenicol.

26. A method according to claim 23 wherein the antibiotic is administered by a route selected from the group consisting of sublingual, transmucosal, transdermal, parenteral, and oral.

27. A method according to claim 23 wherein the antibiotic is a radioactive antibody specific for F. nucleatum or T. denticola.

28. A method according to claim 27 wherein the antibody is conjugated to an agent selected from the group consisting of Astatine-211, Iodine-131, Iodine-125, Yttrium-90, Rhenium-186, Rhenium-188, Samarium-153, Bismuth-212, Phosphorus-32, Lead-212 and Lutetium-177.

29. A method for treating chronic adenotonsillar hypertrophy and tonsillitis comprising removing “sulfur granules” from hypertrophic tonsils.

30. A kit for diagnosing chronic adenotonsillar hypertrophy and tonsillitis comprising an agent capable of detecting the presence of one or more bacteria selected from the group consisting of F. nucleatum and T. denticola in a biological sample.

31. A kit according to claim 30 wherein the agent is an antibody capable of detecting an antigen selected from the group consisting of a F. nucleatum or T. denticola cell, a protein or polypeptide secreted by F. nucleatum or T. denticola and an antibody specific for F. nucleatum or T. denticola.

32. A kit according to claim 30 wherein the agent is an antibody labeled with a fluorescin or a radioactive atom.

33. A kit according to claim 30 wherein the biological sample is selected from the group consisting of blood, saliva and tonsillar tissue.

34. A vaccine for preventing chronic adenotonsillar hypertrophy and tonsillitis due to F. nucleatum or T. denticola comprising an agent capable of inducing an immune response to F. nucleatum or T. denticola.

35. A vaccine according to claim 34 wherein the agent capable of inducing an immune response to F. nucleatum or T. denticola is selected from the group consisting of an antigen of Fusobacterium or Treponema, a whole F. nucleatum or T. denticola cell, a protein or peptide secreted by F. nucleatum or T. denticola, and DNA encoding a protein or polypeptide produced by F. nucleatum or T. denticola.

36. A vaccine according to claim 35 wherein the whole F. nucleatum or T. denticola cell is either killed or attenuated.

37. A vaccine according to claim 35 wherein the DNA encoding a protein or polypeptide produced by F. nucleatum or T. denticola is present on a recombinant vector.

38. A vaccine according to claim 34 further comprising a suitable carrier or adjuvant.
